(19) United States
(12) Patent Application Publication
(10) Pub. No.: US 2023/0100537 A1
(43) Pub. Date: Mar. 30, 2023

(54) SYSTEM AND METHODS FOR CLOUD-BASED INTERACTIVE GRAPHIC EDITING ON ECG DATA

(71) Applicant: ZBeats, Inc., Stony Brook, NY (US)

(72) Inventors: Peng Zhang, South Setauket, NY (US); Bin Fang, Setauket, NY (US)

(73) Assignee: ZBeats, Inc., Stony Brook, NY (US)

(21) Appl. No.: 17/800,097

(22) PCT Filed: Feb. 23, 2021
(86) PCT No.: PCT/US2021/019243

§ 371 Date: Aug. 16, 2022

Related U.S. Application Data
(60) Provisional application No. 62/980,883, filed on Feb. 24, 2020.

(51) Int. Cl. G16H 15/00; G16H 40/60

(52) U.S. Cl. CPC G16H 15/00; G16H 40/60; G16H 50/20

(57) ABSTRACT

Systems and methods for obtaining, storing and analyzing electrocardiogram (ECG) data utilizing the cloud virtualized unlimited resources to aid in the process of detection of arrhythmia on ECG data. It also comprises various cloud-based interactive graphic editing tools, to augment medical practitioners' capabilities in the course of inspecting and editing any misinterpretations. This system captures the ECG data from ECG devices to databases utilizing cloud communication and stores in various storage modules such as ECG signal data, record annotation, and patient's information. The cloud-based interactive graphic editing tools comprise: representation and visualization of ECG data; examining the data to decide parameters for computer-aided diagnosis (CAD) programs; human adjustment of detected QRS fiducial points and R points; interactive visualization, classification of waveform based demix graphs, R-R and heart-rate time-series and histograms, and the scatterplots for classified R-R intervals in time domain.

### CROSS REFERENCE TO RELATED APPLICATIONS

This application claims priority to U.S. Provisional Application No. 62/980,883 filed Feb. 24, 2020, titled “SYSTEM AND METHODS FOR CLOUD-BASED INTERACTIVE GRAPHIC EDITING ON ECG DATA”, the contents of which are incorporated herein by reference.

### FIELD

The present disclosure relates to a system for electrocardiogram (ECG) data acquisition, processing and storage utilizing cloud virtualized resources, and supported cloud-based interactive graphic editing tools.

### BACKGROUND

The following description includes information that may be useful in understanding the present invention. It is not an admission that any of the information provided herein is prior art or relevant to the presently claimed invention, or that any publication specifically or implicitly referenced is prior art.

The electrocardiogram (ECG) is a graphic representation of the electrical activity of heart. It is considered as a fundamental, effective tool of clinical practice as it is a simple, non-invasive exam that is performed by a health professional.

For example, if a patient has signs or symptoms of a heart problem or unexplained fainting, a doctor may order an ECG test, to check the patient's heart activity and detect any irregularities in the heart rhythm.

A standard Holter monitor is often used to record a continuous 24 to 48-hour's heart electrical activity (ECG). This information is used to detect if a patient has an arrhythmia and to monitor how frequently it happens. A 24 to 48-hour ECG test is referred to as a short-term ECG test.

However, sometimes such short-term ECG test cannot give enough information to detect any irregularities because the patient is hooked up to the machine for only a short time. This results in a low diagnostic yield, with 35% in arrhythmias in general and even lower in syncope patients.

In patients with infrequent symptoms, the duration of continuous ambulatory ECG monitoring has been increased from 24-48 hours monitoring to a range of 7-30 days ECG monitoring, in order to increase diagnostic yield significantly.

The newer Holter monitor including an adhesive Holter patch, can continuously record and store data for two weeks (14 days) or even longer, this leads to a higher diagnostic yield, but also results in large amounts of retrieved data that need to be analyzed. Another type of long-term ambulatory ECG method is the 30-day, near real-time mobile cardiac telemetry (MCT) monitors. This provides in-time feedback to the cardiologist, while the patient is still being monitored.

As required monitoring time increases, diagnostic burden is becoming more severe as it appears more related to the amount of ECG data that needs to be analyzed. Today, most of these companies and manufactures still use personal computer-based software to do the ECG data acquisition, storage and editing tasks on users' personal computer (PC). Such system and tools have many fundamental limitations. Such ECG editing computer must have the required processing power and memory to review and edit the ever-increasing amounts of ECG data, and provide sufficient diagnostic information to a user (ECG technician, cardiologist). A typical PC might handle up to 7-10 days ECG strips. However, any longer ECG monitoring such as 14 to 30 days records requires additional memory space that exceeded the memory capability of a typical PC. Adding to memory limitation, the processing power limitation on PC software is more serious as scalability is hardly achievable on PC systems.

Recently, some approaches use a centralized data storage system that enables data upload and download from geographically dislocated offices. But this is not a cloud-based system. Such system still relies on user's local computer processing to do the ECG editing and thus the memory space and processing power limitations are not addressed. Moreover, this solution increases the possibilities of a hacker accessing and modifying patient's data, when data is transforming between centralized storage and local computers.

The need exists for a more reliable and efficient system to aid a technician and doctor in the process of identifying cardiac arrhythmias, precisely differentiating morphological features, and even characterizing transient morphological changes. A system according to the invention principles addresses these problems.

### SUMMARY

Embodiments of the present disclosure aim to devise a cloud-based system that acquires data through using cloud communication, stores the data using various cloud virtualized limitless resources, processes the data through cloud-based interactive graphic methods with users, and creates a report.

In a data acquisition process, the system offers two options for data loads: manual and automatic. The manual method is intended to be used by a medical practitioner to upload the data from the memory of an ECG device to the cloud server. The automatic method is intended to be as an Application Programmable Interface (API) that allows a program or device to automatically upload data to a cloud server.

In a data diagnostic process, the system offers an integrative framework by combining human and artificial intelligence (AI), to improve the efficiency for clinical staff. An AI-enabled algorithm is used for the beat classification, the waveform-based clustering and the detection of arrhythmia. In-between those automated steps, users are allowed to inspect intermediate results and adjust any misinterpretations through some provided interactive graphic tools. The proceeding automated steps are capable to adjust themselves based on human input. This design is to make sure ECG data interpreted in an accurate and efficient way by humans.

Technical advantages of ECG analysis system include increased accuracy in analysis of ECG data. For example, by providing interactive user interfaces including editing capabilities, a user can edit the features that are input into a neural network algorithm for cardiac rhythm classification. As a result, accuracy of cardiac rhythm classification is increased. Further, the interactive user interface allows the clinician to view, inspect, and/or edit data at various stages in the processing pipeline, which allows the clinician to not only identify any errors in the processing the ECG signal but also to make necessary edits, which improves ECG classification. Further, the ability to view, inspect, and/or edit improves clinician confidence in diagnosis, which in turn improves patient outcomes. Furthermore, by adapting a cloud-based approach, patient ECG data may be acquired over a long duration of time and processed with increased accuracy. Further still, changes to ECG feature annotations are tracked, which enables the clinician to track changes made by another clinician, which may facilitate a novice clinician to learn ECG analysis.

### BRIEF DESCRIPTION OF THE DRAWINGS

The present disclosure, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

FIG. 1 is a schematic illustration showing various components of the cloud-based system utilizing various cloud virtualized resources, and the cloud communication between various client modules and cloud server modules.

FIG. 2 illustrates the steps of the overall methodology for use by a technician or clinical staff to process ECG signal data, and detect abnormalities of ECG beats and rhythms, and generate the report.

FIG. 3 is an illustration of a normal ECG curve comprising of two consecutive QRS complexes, where important intervals and points of measurement are depicted.

FIG. 4 illustrates the interactive visualization method for the ECG data where the R-wave peak positions are allowed to be edited and adjusted manually by technicians and clinical staff.

FIG. 5 is an illustration for overlapping multiple ECG waveforms, aligned by detected R-wave peaks. The beat-level information such as classification and template can be adjusted. The individual ECG waveforms are represented.

FIG. 6 is an illustration of a scatter plot for classified R-R intervals in time domains. A pair of successive two R-R intervals is used as x- and y-axis values, respectively, for a QRS complex. A selection tool based on regions is provided. Adding to the scatter plot, a side section is used to show waveforms for selected QRS complexes.

FIG. 7 is an illustration of a histogram for classified R-R intervals in time domains. In the histogram, the R-R intervals are first classified as categories then group each category by bins. The height of each bar shows how many R-R intervals of a category fall into a bin range. Interactively, a downside section shows waveforms of selected bin range, that allow manual online editing.

FIG. 8 is an illustration of a time series of all R-R intervals, also named as a trend plot of R-R intervals time series. This plot intuitively shows the variation in the interval between detected heart beats known as R-R intervals. Interactively, a downside section shows waveforms of QRS complexes in selected regions, that allow manual online editing.

FIG. 9 illustrates combination of (1) time series of R-R intervals, and (2) overlapping of multiple ECG waveforms. This method uses the time series of R-R intervals as an interactive tool to select segments of ECG waveforms. Selected ECG waveforms are overlapped, aligned by their R-wave peaks. Merging and splitting templates are allowed, as well as adjusting categories for selected ECG beats.

FIG. 10 illustrates combination of (1) time series of R-R intervals, and (2) a scatter plot of classified R-R intervals. This method uses the time series of R-R intervals as an interactive tool to select segments of ECG waveforms. A scatter plots for these selected ECG waveforms is shown.

FIG. 11 illustrates the representation of these detected and classified arrhythmias. Three sections are shown for a series of rhythm classes, the waveforms for cardiac arrhythmias of a class, and the position of a cardiac arrhythmia in the original ECG signal time series. Interactive tools are provided to allow human editing.

### DETAILED DESCRIPTION

### An Example of the Cloud-Based System Architecture

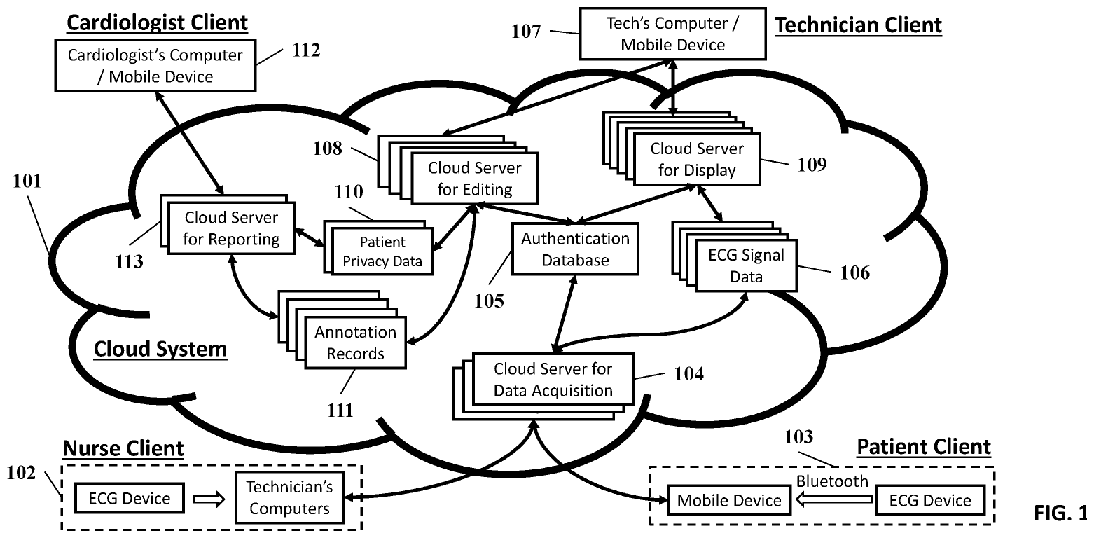

FIG. 1 Illustrates an exemplary overall cloud system architecture according to one embodiment of the present disclosure. In the embodiment shown in FIG. 1, a plurality of two different types of data acquisition methods, 102 and 103, are shown for uploading ECG data from remote locations and devices via the internet to the “cloud” system 101. A cloud system refers to as a system with various virtualized resources, especially data storage and computing power, for enabling the delivery of cloud computing services via a network.

### Data Acquisition to Cloud

In the embodiment shown in FIG. 1, the nurse client 102, is one example of data uploads for being used by a medical practitioner such as a nurse practitioner. In one example, after wearing an ECG device such as a Holter monitor, the patient returned the device to the doctor's office where the stored ECG signal data is transmitted via a technician's computer to a cloud server 104. The cloud server 104 comprises one or more processors and one or more memories storing executable instructions for classifying irregular heart rhythms, as discussed below. A web browser can be used for a user to create a patient record and upload ECG signals to the record on server 104. This is a client-server model, in which the client is the nurse client 102 and the server is a cloud server for data acquisition 104.

In the embodiment shown in FIG. 1, the patient client 103, is one example of automatic or semi-automatic data uploads for being used in a large variety of ECG devices. These ECG devices can be ambulatory ECG recording devices such as a Holter monitor, an adhesive Holter patch, an ECG sensor, an event recorder or a mobile telemetry. In the example, an ECG device (e.g., an ECG device including ECG sensors) acquiring ECG signals from a patient, and communicates by a wired or wireless communications network, such as Bluetooth or Bluetooth Low Energy (BLE), to a mobile device (e.g., a mobile phone) carrying an application that buffers and uploads the data to the server 104 through a communications network, such as cellular, ZigBee, or Wi-Fi networks. The mobile device may also perform some rudimentary analysis of the incoming ECG signals, for example, to check for emergencies and directly send alert to a clinical staff when an emergency detected. An emergency can either be generic or predefined arrhythmia event type and duration for a specific patient by the patient's ordering physician. The patient client 103 can upload the incoming data in a fixed time of period or patient-triggered or algorithm-triggered or a combination of some data transmission policies.

Generally speaking, two types of clients 102 and 103 are shown to transmit ECG signals from the devices to the cloud system 101. The commutation between the clients (102, 103) and the server 104 can be through a Wi-Fi network, direct connection to the internet, a mobile data network, a cellular network, direct connection to phone lines or any other type of communication that allows data transmission between a client and a cloud server to exist.

### Cybersecurity in Cloud

The cloud 101 includes an authorization module 105 which specifies access rights and privileges to cloud resources. In one example, to upload data to the cloud server, an application in mobile device 103 must pass authorization check. Another example, to access services provided in the cloud 101, any user must pass authentication and authorization checks. These user and device credentials are stored in the database 105.

### Data Storage in Cloud

Adding to data acquisition, the cloud 101 utilizes various virtualized resources to store the data. In the embodiment shown in FIG. 1, the ECG signal data stored in 106, the patient privacy information (e.g., name, gender, age) stored in 110, and the annotation information (e.g., R-wave peaks, classification, template, detected arrhythmias) stored in 111. This cloud-based storage solution separates sensitive user information from patient's ECG signal data, to enhance the user privacy.

The data storage solution further allows separation of privileges to various operational modules. A cloud server for data acquisition 104 is only allowed for appending to ECG signal data 106. The cloud server for display 109 is granted to access the ECG signal data in a read-only mode. The cloud server for editing 108, which uses a separate link, can access patient privacy data 110 and these annotation records 111 with a write permission. The cloud server for reporting 113 is allowed to read the patient privacy data and the annotation records.

### Technician Access to Cloud

In the embodiment shown in FIG. 1, a technician in 107 refers to a human expert who can perform editing actions (read data, examine, add, delete and modify annotations). The editing actions are performed through utilizing provided interactive graphic editing tools in cloud system 101. From the clinical point of view, a technician can be a telemetry nurse or expert cardiologist or a professional who is qualified and certified to perform the ECG diagnostic process.

A technician client 107 can access the cloud system 101 through using two servers: a cloud server for display 109 and a cloud server for editing 108. The cloud server 109 provides visualization operation through read-only access to the ECG signal data module 106. The cloud server for editing 108 grants the read-and-write accesses to both the patient's privacy data and the annotation records. These interactive editing tools are utilizing the operational module 108, and the visualization is through the module 109.

### Cardiologists Access Reports

In the embodiment shown in FIG. 1, the last client module is the cardiologist client 112. This client module is designed reviewing and obtaining a report. In the client module 112, a “cardiologist” (e.g., an ordering physician or cardiologist or any other doctor) can review a report through a web browser, or obtain an electronic copy of the report, or download an electronic copy to local computers.

### Methodology Steps

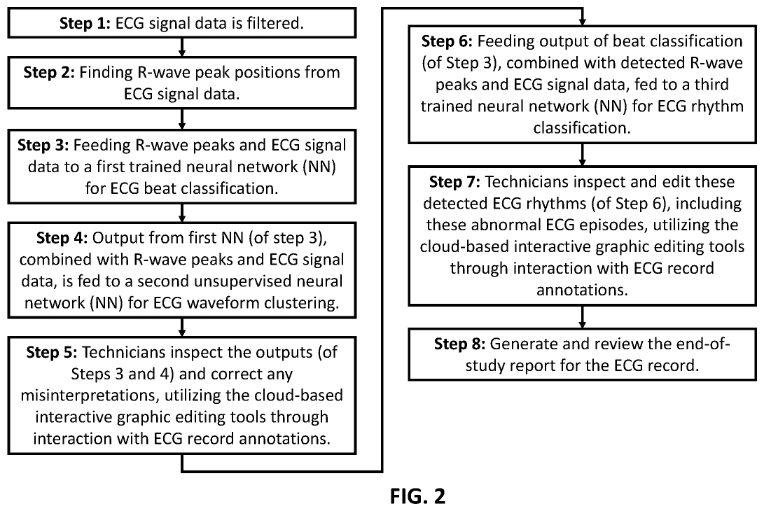

FIG. 2 illustrates the steps of the overall methodology for detecting and classifying arrhythmias in the ECG signal data according to one embodiment of the present disclosure. The method of FIG. 2 may be performed by one or more processors in the cloud system 101 according to executable instruction stored therein. The steps are described in detail as follows:

In Step 1: the incoming ECG signal data from ECG devices is filtered by a combination of several filtering techniques. The main purpose of the step is to apply denoise and baseline wandering removal on the raw ECG signal. One example is that, using an adaptive Least Mean Squares (LMS) filter, followed by an adaptive Discrete Wavelet Transformer (DWT) with “soft” threshold on SureShrink for all the detail coefficients. These filtered ECG signal data stored in the module 106 of FIG. 1.

In Step 2: the filtered ECG signal data (of Step 1) is fed into an algorithm (e.g., the Pan-Tompkins algorithm) that is used to detect R-wave peaks, also names R-peaks, of QRS signals. These detected R-peaks stored as annotation file in the module 111 shown in FIG. 1.

In Step 3: the detected R-peaks (of Step 2) and the filtered ECG signal data (of Step 1) are fed into a first trained neural network (NN), for ECG beat classification.

In one embodiment, the ECG beats can be classified into five categories: supraventricular ectopic beats (S), ventricular ectopic beats (V), unclassified beats (X), normal beat (N) and artifacts (A). A supraventricular ectopic beat, labeled as S, can be an atrial or nodal (junctional) premature or escape beat. A ventricular ectopic beat, labeled as V, can be a ventricular premature beat or escape beat. A “normal” beat, labeled as N, can be a normal or bundle branch block beat. An unclassified beat, labeled as X, can be a paced beat, a fusion of a packed and normal beat, or a beat that cannot be classified. An “artifact” beat, labeled as A, is any beat that does not fall into the S, V, X, or N categories described above. The categories of heartbeats are not restricted to these five categories in this embodiment.

In Step 4: these classified ECG beats (of Step 3) are fed into an unsupervised machine learning method for ECG waveform clustering. Clustering is the process of grouping ECG beats with similar morphologies together. A “group” is also named as a “template”. In one embodiment, K-means clustering can be applied, with the ECG waveforms as inputs, to create templates. The output of Step 4 is the “templates” for ECG beats, and stored as annotation file in the module 111 as shown in FIG. 1.

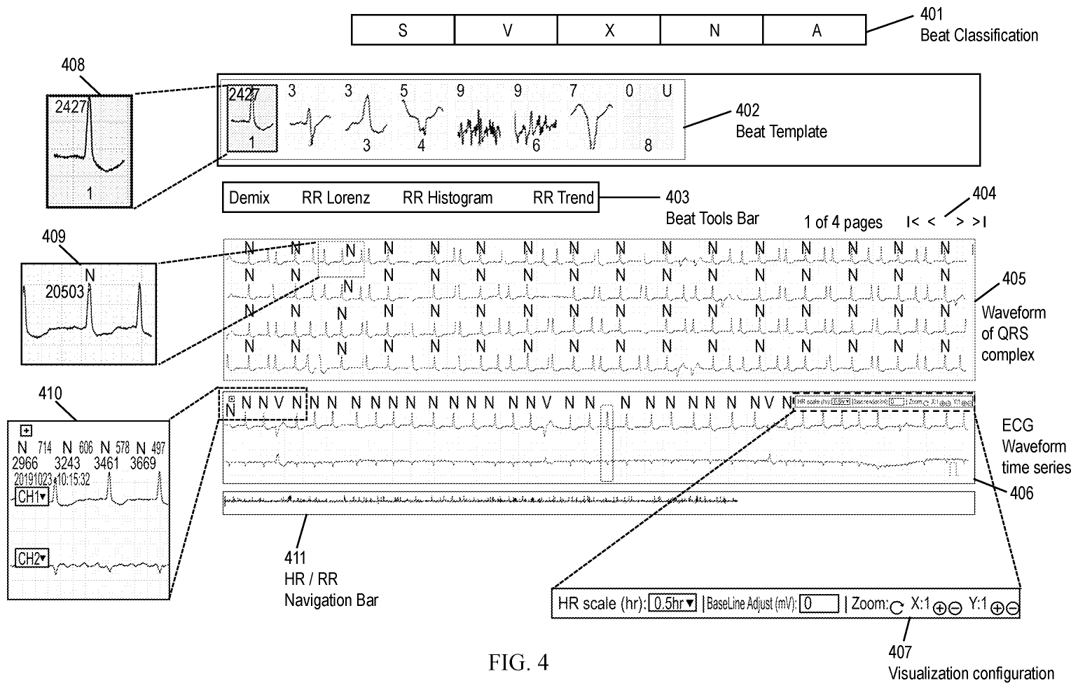

The outputs of Steps 2, 3 and 4 are the detected R-wave peaks, the categories of ECG beats, and the templates of each beat categories are together displayed via an interactive graphical user interface as shown in FIG. 4.

In one embodiment, a technician starts to inspect these outputs (of Steps 2, 3, and 4) and correct misinterpretation. Any editing operation is completed through utilizing these interactive graphic tools as shown in FIGS. 4, 5, 6, 7, 8, 9 and 10. The preferred embodiments for the interactive graphic editing tools will be in detail described in the section of “AN EXAMPLE OF THE CLOUD-BASED INTERACTIVE GRAPHIC EDITING TOOLS”.

All the annotation editing operations will be recorded in annotation records 111 as shown in FIG. 1. These said editing operations include:

(1) Editing R-wave peaks: add, delete and adjust R-wave peaks of QRS complexes.

(2) Editing beat category and template: change the category and template of QRS beats.

In Step 6: these inspected outputs (which may include viewed and/or modified outputs) of R-peak waves (of Step 2), the beat category (of Step 3) and the beat template (of Step 4), with the ECG signal data, are fed into a third trained neural network (NN) model for the detection and classification of irregular cardiac rhythms or runs. Said another way, edited outputs (which include outputs modified and/or viewed by an authorized healthcare provider (e.g., clinician, technician etc.) are used as input into the third neural network algorithm trained for detection and classification of irregular cardiac rhythms or runs.

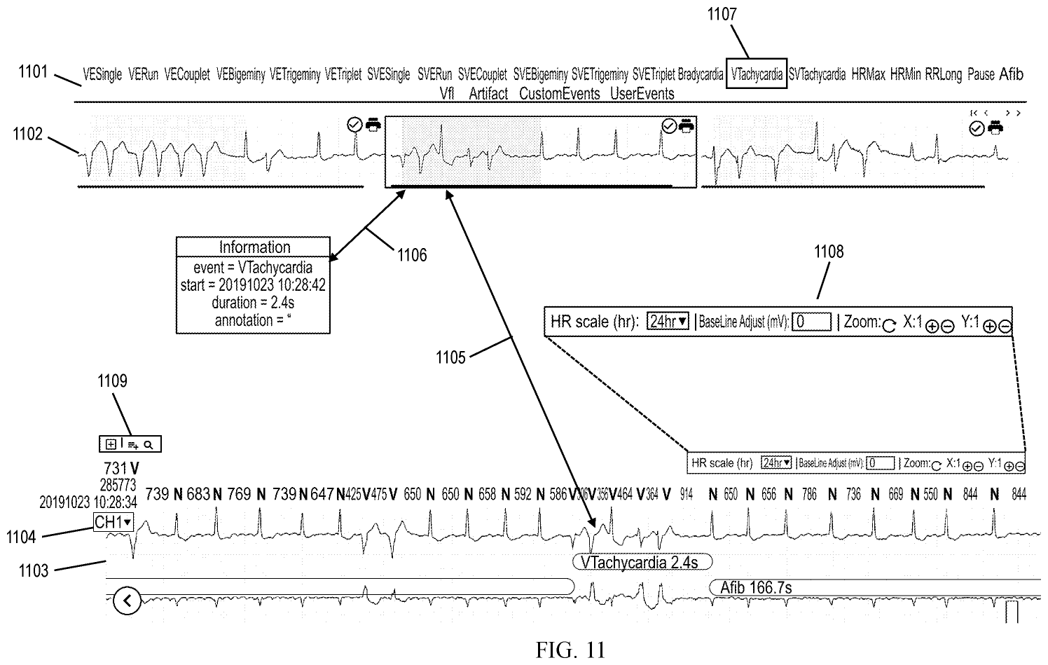

In Step 7: the outputs (of Step 6) are displayed as shown in FIG. 11. A technician can edit these detected and classified rhythms. For example, a second interactive graphical user interface is generated and displayed to the user for editing the detected and classified rhythms before generating a report. These editing functions in FIG. 11 will be in detail described in the section of “AN EXAMPLE OF THE CLOUD-BASED INTERACTIVE GRAPHIC EDITING TOOLS”.

### An Example of the Cloud-Based Interactive Graphic Editing Methods

FIGS. 4 to 10 are the interactive graphic editing methods for single ECG beats.

FIG. 11 is an interactive graphic editing method for ECG runs.

Visualization method in FIG. 4:

FIG. 4 illustrates the virtualization of the detected and classified ECG beats. It is a web browser as a graphical user interface of interacting with ECG annotation records.

In certain embodiment, the design of FIG. 4 comprises the following sections:

Section 401 of FIG. 4 is beat categories. Each letter or phrase represents a beat category.

Section 402 of FIG. 4 is beat templates. Each small plot in this section shows a particular beat template within the category, and is named as a template icon 408. A typical template icon 408 includes an ECG waveform for that template, the total number of ECG beats in that template (e.g., 2427 in top left corner), and the template serial number (e.g., 1 in bottom center), as shown in FIG. 4.

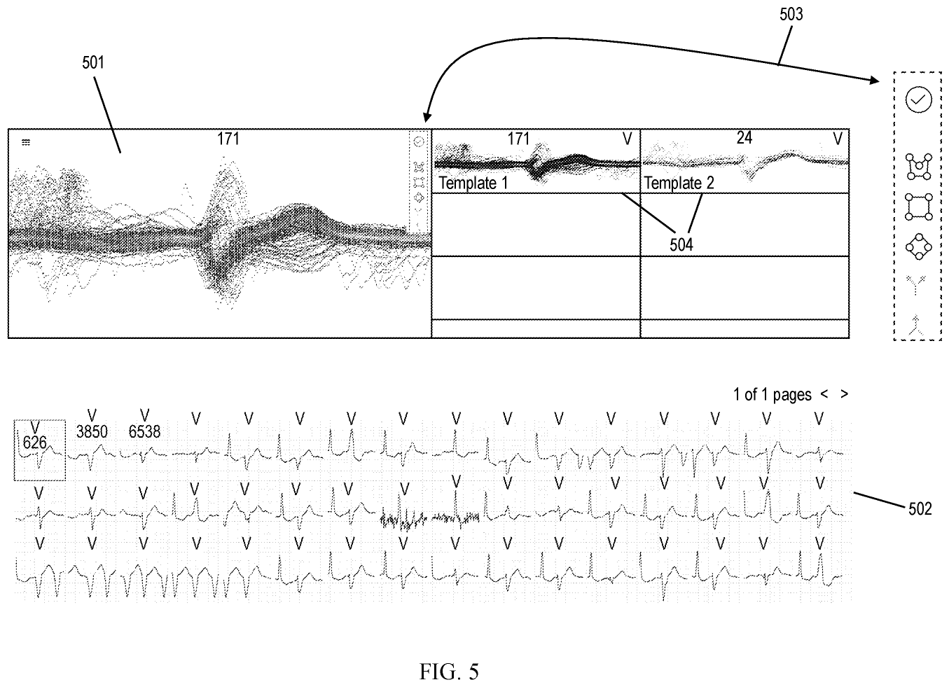

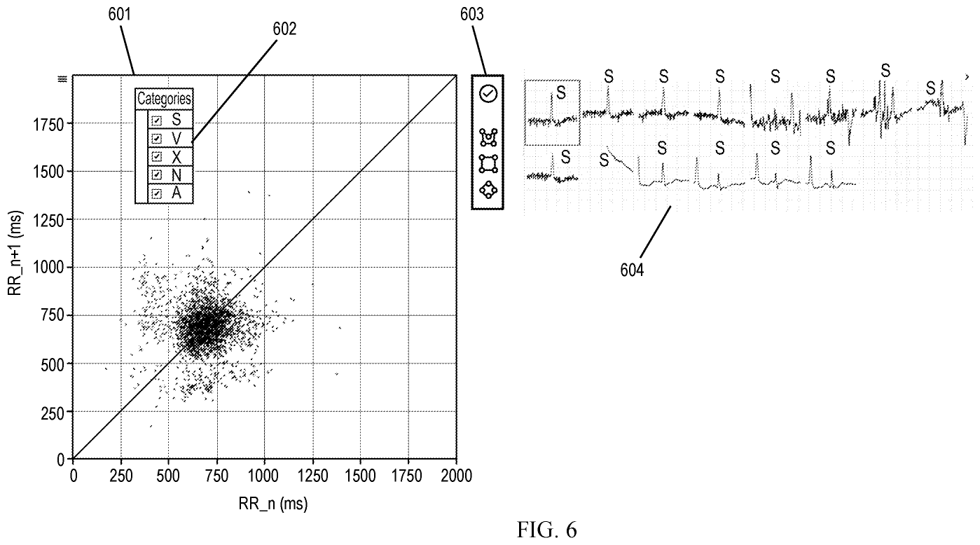

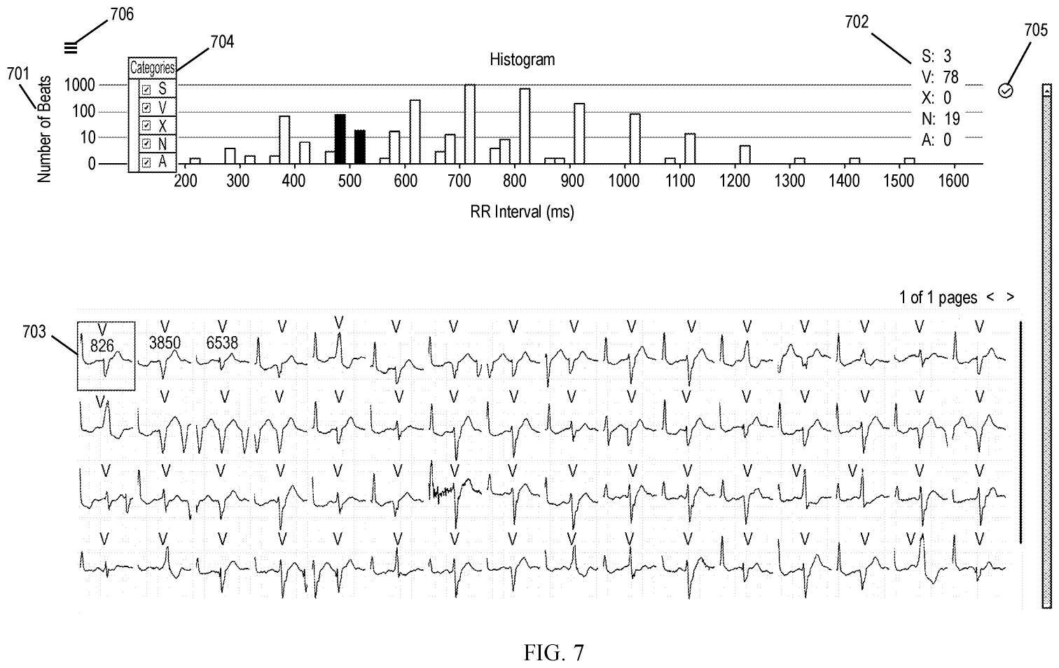

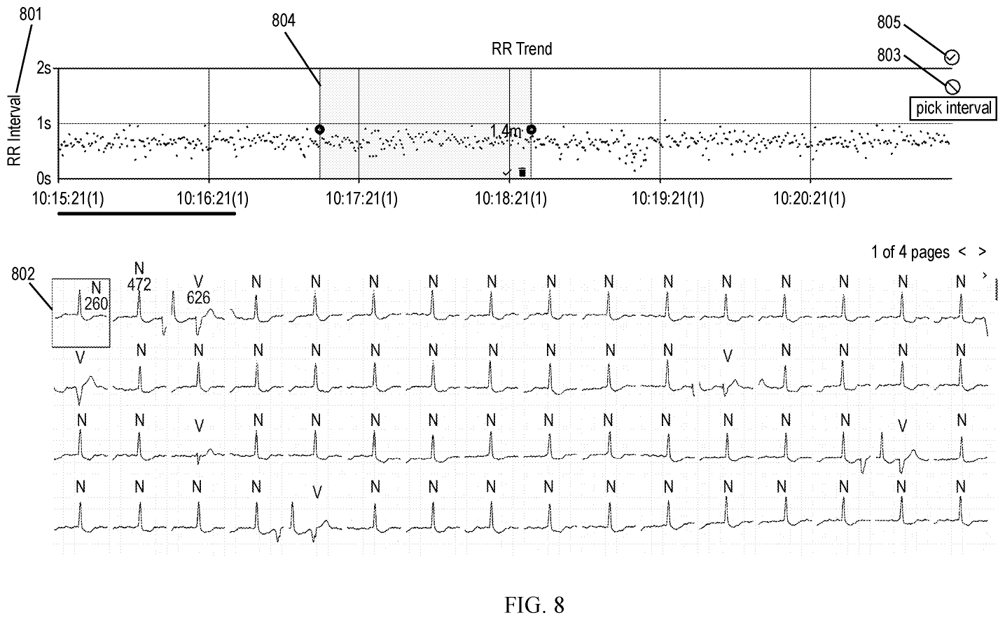

Section 403 of FIG. 4 is a tool bar to show additional interactive tools. These tools include Demix (shown in FIG. 5), RR Lorenz (shown in FIG. 6), RR Histogram (shown in FIG. 7), and RR Trend (shown in FIG. 8). These graphic interactive tools help a user to visualize, inspect and edit the ECG beats classification quickly.

Section 404 of FIG. 4 is a quick view tool bar including a page number and total pages, a pair of forward and rewind buttons, and a pair of fast-forward and fast-rewind buttons.

Section 405 of FIG. 4 is a window section comprising multiple individual ECG waveforms of fixed time length. 409 is a window for one ECG waveform, in which the waveform is shown, the category letter “A” of this beat is shown in the top center, the serial number “20603” of this QRS complex is written below the beat category. In certain embodiment, the serial number can be ignored or replaced by a time stamp.

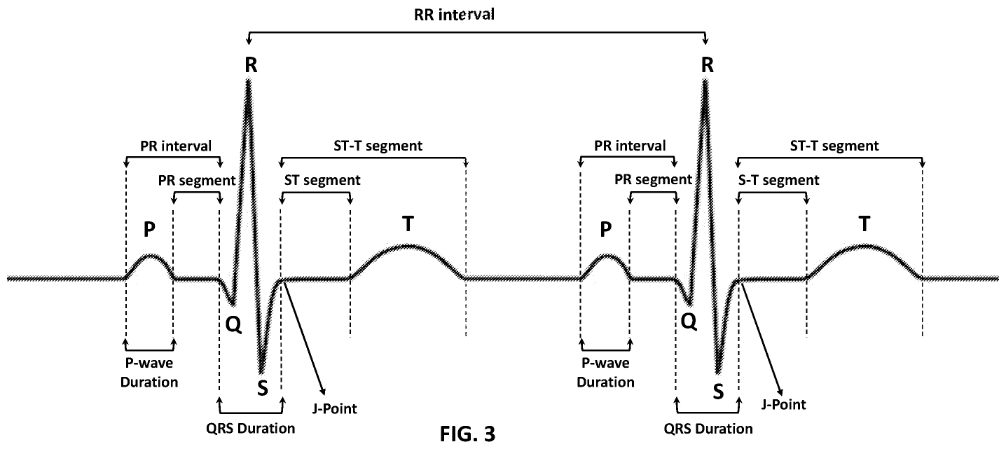

Section 406 of FIG. 4 is a window to show the ECG signals time series of multiple channels. In this example, two time series are shown for two channels (e.g., MLII, V1). 410 is a zoomed screenshot to show local properties. A category letter is marked above R-wave peak. A number between two adjacent category letters refers to the time interval between two consecutive R-wave peaks, namely the length of R-R interval (as shown in FIG. 3). The number below a category letter is the serial number of the R-wave peak. 407 is a visualization configuration toolbar for section 406. In 407, a user can adjust the heart-rate (HR) scale, adjust the baseline (y-axis shift), zoom the waveform in x-axis (time scale) or in y-axis (signal scale).

Section 411 of FIG. 4 is HR/RR navigation bar. HR refers to the heart rate in BPM (beats per minute). RR refers to the time interval between two successive R-wave peaks as shown in FIG. 3. The formula between the HR (in BPM) and the RR (in seconds) is HR=60/RR. This navigation bar shows a time series of HR or RR values.

Six interactive editing methods in FIG. 4:

(4.1) in section 402 of FIG. 4, a user can re-categorize a whole template to a new beat category by pressing a key letter. For example, select a template and press a letter “S” then all QRS complexes of that template are categorized as supraventricular ectopic beats.

(4.2) in both section 405 and section 406 of FIG. 4, a user can select a single or multiple QRS complexes then press a key letter to re-categorize them, like (4.1).

(4.3) in both section 405 and section 406 of FIG. 4, a user can delete misidentified single or multiple QRS complex by highlighting them and clicking DEL.

(4.4) in section 406 of FIG. 4, a user can add a missing R-wave peak: click “+” in section 410, choose a region using the mouse and then add R-wave peaks by pressing a key letter to indicate the beat category, and the system will automatically put the beat(s) into the most suitable template(s).

(4.5) in section 406 of FIG. 4, a user can delete a R-wave peak: choose the R-wave peak and click “−” to delete the chosen R-wave peak.

(4.6) in section 406 of FIG. 4, a user can adjust the position of a R-wave peak: click the R-wave peak and drag it to the target position.

All the editing operations (4.1) to (4.6) in FIG. 4 are through the cloud server 108 and updated the annotation records in 111 of FIG. 1.

Visualization method in FIG. 5:

FIG. 5 illustrates an interactive method of overlapping multiple ECG waveforms in graph. This graph is also called a Demix graph. A Demix graph allows overlays of detected and classified ECG signal waveforms. All ECG signal waveforms are aligned by detected R-wave peaks. The color at any pixel of the graph represents the density of overlapped ECG waveforms.

Two interactive editing methods in FIG. 5:

(5.1) template splitting: a user can select a region of interest in 501 by clicking and dragging single or multiple shapes from session 503, and then press “split” icon to split the QRS complexes in selected region as a new template from the rest of QRS complexes in original template.

(5.2) template merging: a user can select multiple templates in 504 and then press “merge” icon to merge all these templates into one.

All the editing operations (5.1) to (5.2) in FIG. 5 are through the cloud server 108 and updated the annotation records in 111 of FIG. 1.

Visualization method in FIG. 6:

FIG. 6 is an illustration of a scatter plot for R-R intervals, also name as R-R Lorenz plot. A pair of successive two R-R intervals (shown in FIG. 2) is used as x- and y-axis values, respectively, for a QRS complex. Section 601 shows the scatter plot. 602 is a selection bar for five categories of ECG beats, allowing a user to easily select or deselect which categories the user wants to show in the scatter plot and to arrange the category showing order in the plot. Section 603 is a region selection toolbar, allowing a user to select multiple regions of interest in the scatter plot. All ECG complexes that fall in these selected regions are shown in the section 604. Of them, every ECG complex is shown in a small window, in which the letter and number are the category and serial number of the QRS complex, respectively.

Two interactive editing methods in FIG. 6:

(6.1) in Section 601 of FIG. 6, a user is allowed to select multiple regions of data points, and then press a key associated with a category (e.g., “S”), and all selected QRS complexes falling in the regions are re-categorized (that is, re-classified by user editing) as supraventricular ectopic beats (namely, “S”). Simultaneously, this operation is completed on corresponding annotation records 111 (of FIG. 1) through the cloud server for editing 108 (of FIG. 1). Upon the completion of this change on annotation records 111, sections 601 and 604 of FIG. 6 are updated.

(6.2) in Section 604 of FIG. 6, a user is allowed to select single or multiple QRS complex complexes, and then press a key associated with a category (e.g., “S”), and last all selected QRS complexes are re-categorized as supraventricular ectopic beats (namely, “5”). Simultaneously, this operation is completed on corresponding annotation records 111 (of FIG. 1) through the cloud server for editing 108 (of FIG. 1). Upon the completion of this change on annotation records 111, sections 601 and 604 of FIG. 6 are updated.

All the editing operations (6.1) to (6.2) in FIG. 6 are through the cloud server 108 and updated the annotation records in 111 of FIG. 1.

Visualization method in FIG. 7:

FIG. 7 illustrates a histogram of classified R-R intervals. In certain embodiment, these R-R intervals are first classified into beat categories and then grouped into bins to create a histogram. Section 701 shows a histogram, where the height of each bar shows how many R-R intervals of a category fall into the bin range. As a mouse over a region of 701, 702 is a bar to show the number of beats in each category. 704 is a selection bar that allows a user to select or deselect which categories to be considered. When a user chose a particular “bar” in the histogram section 701, the ECG complexes belonging to that bar, are shown in section 703. Of them, every ECG complex is shown in a small window, in which the letter and number represent the category and serial number of the QRS complex, respectively. 705 is a return button to FIG. 4. 706 is a flash button for creating a new histogram.

Two interactive editing methods in FIG. 7:

(7.1) In Section 703 of FIG. 7, a user first selects one or multiple ECG complexes, and then presses a key associated with a target category (e.g., “S”), and last all the selected ECG complexes are recategorized as supraventricular ectopic beats (namely, “S”). Simultaneously, this operation is completed on the corresponding annotation records 111 (of FIG. 1) through the cloud server for editing 108 (of FIG. 1). Upon the completion of this change on annotation records 111, sections 701 and 703 of FIG. 7 are updated accordingly.

(7.2) In Section 701 of FIG. 7, a user can select a bar of the histogram and then press a key associated with a target category (e.g., “S”), and all the ECG complexes in that bar are recategorized as supraventricular ectopic beats (namely, “S”). Simultaneously, this operation is completed on the corresponding annotation records 111 (of FIG. 1) through the cloud server for editing 108 (of FIG. 1). Upon the completion of this change on annotation records 111, sections 701 and 703 of FIG. 7 are updated accordingly.

All the editing operations (7.1) to (7.2) in FIG. 7 are through the cloud server 108 and updated the annotation records in 111 of FIG. 1.

Visualization method in FIG. 8:

FIG. 8 is an illustration of a time series of all R-R intervals. In one embodiment, the chart has two sections: section 801 shows the time series of R-R intervals, where x-axis is the time axis and y-axis are the R-R intervals; section 802 shows all the QRS complexes within a selected time period. By clicking button 803, a user calls the function for time period selection, as shown in 804. 805 is a return button to FIG. 4.

One interactive editing method in FIG. 8:

(8.1) User can pick and highlight a time period in Section 801 of FIG. 8, as shown in section 804. All QRS complex falling in the time period, regardless of their categories will be shown in section 802. User can select one or multiple QRS complexes in section 802, and press a key associated with a target category (e.g., “S”), and all the ECG complexes in that bar are recategorized as supraventricular ectopic beats (namely, “S”). Simultaneously, this operation is completed on the corresponding annotation records 111 (of FIG. 1) through the cloud server for editing 108 (of FIG. 1).

The editing operation (8.1) in FIG. 8 is through the cloud server 108 and updated the annotation records in 111 of FIG. 1.

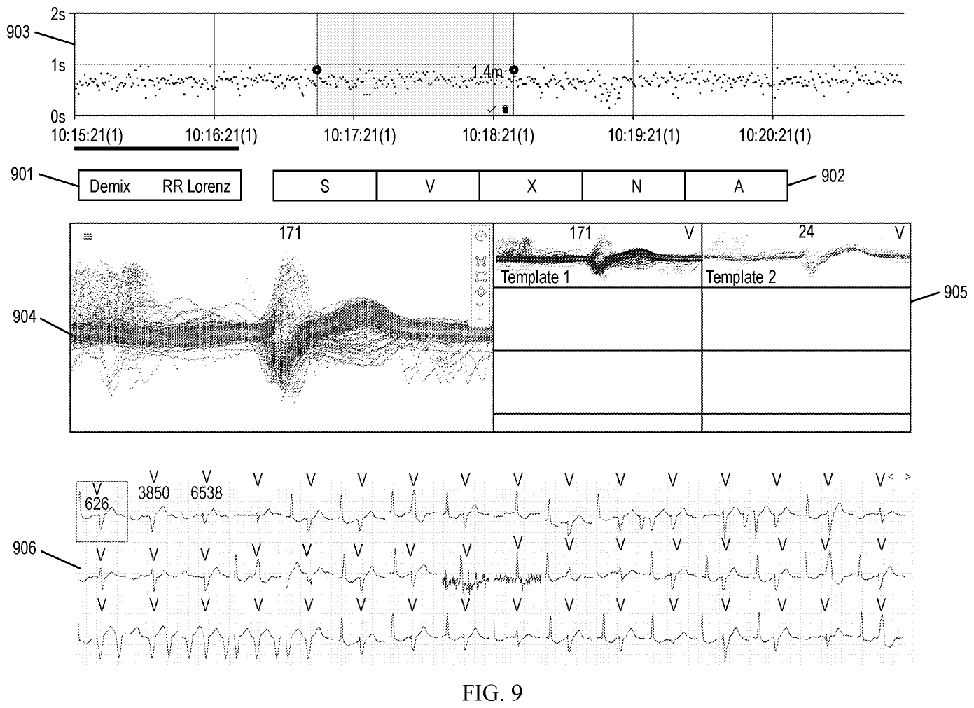

Visualization method of FIG. 9:

FIG. 9 illustrates a composite method. In one embodiment, section 903 is a time series of R-R intervals and serves as a navigation. User selects a time period in 903 and selects certain beat categories in 902. Then, the morphologies of these selected QRS complexes are overlapped in section 904. The user is allowed to merge or split these templates as shown in 905. These QRS complexes overlapped in section 904 are also shown in section 906, where each small window represents a single QRS complex.

Three interactive editing methods in FIG. 9 as follows:

(9.1) in section 903, a user can select a continuous period of time and re-categorize all the QRS complexes in that time period to a beat category by pressing a key associated with the target category (e.g., “S”).

(9.2) in section 905, a user can re-categorize a whole template of QRS complexes to a beat category by pressing a key associated with the target category (e.g., “5”).

(9.3) in section 906, a user can select one or multiple QRS complexes and re-categorize these selected QRS complexes to a beat category by pressing a key associated with the target category (e.g., “S”).

All the editing operations (9.1) to (9.3) in FIG. 9 is through the cloud server 108 and updated the annotation records in 111 of FIG. 1.

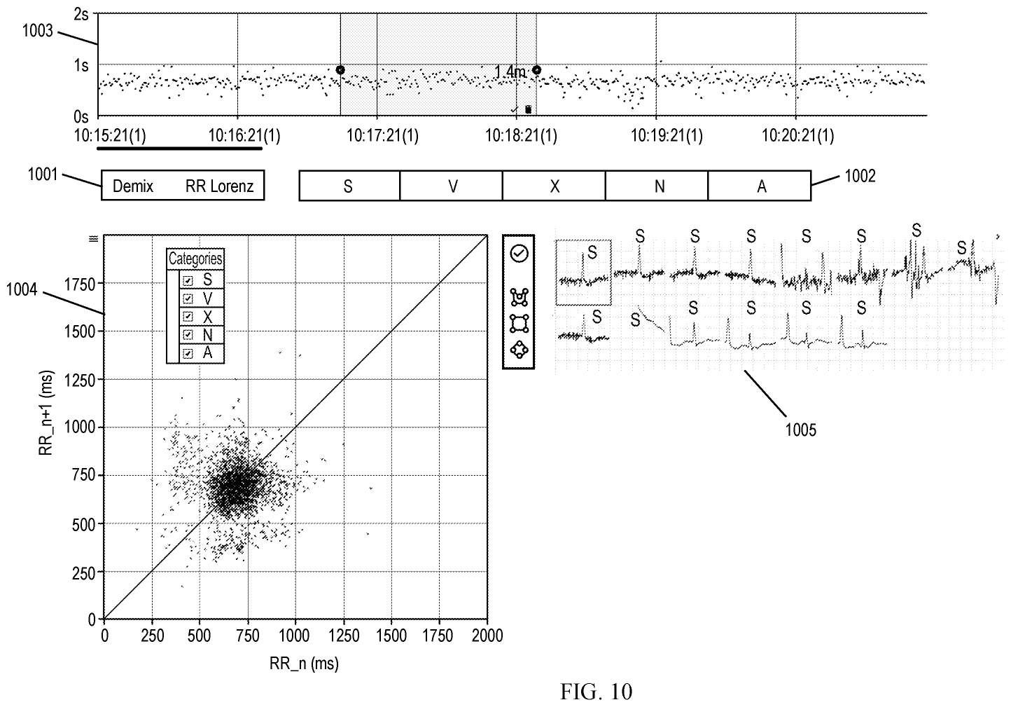

Visualization method of FIG. 10:

FIG. 10 illustrates a composite method. In one embodiment, section 1003 is a time series of R-R intervals and serves as a navigation. A user selects a time period in 1003 and selects certain beat categories in 1002. Then section 1004 shows a scatter plot for selected QRS complexes. A pair of successive two R-R intervals is used as x- and y-axis values, respectively, for each QRS complex. In section 1004, a user allows to select a region of interest. The QRS complexes that fall in that selected region of interest are displayed in the section 1005. Each small window in 1005 shows one QRS complex, in which the letter and number represents the beat category and serial number of that QRS complex.

Three interactive editing methods in FIG. 10 as follows:

(10.1) in section 1003, user can select a continuous period of time and re-categorize all the QRS complexes in that time period to a beat category by pressing a key associated with the target category (e.g., “S”).

(10.2) in section 1005, user can first select a region of interest and then re-categorize all the QRS complexes in that selected region to a beat category by pressing a key associated with the target category (e.g., “S”).

(10.3) in section 1005, user can select one or multiple QRS complexes then re-categorize these selected QRS complexes to a beat category by pressing a key associated with the target category (e.g., “S”).

All the editing operations (10.1) to (10.3) in FIG. 10 are through the cloud server 108 and updated the annotation records in 111 of FIG. 1.

Visualization of ECG irregular rhythms in FIG. 11:

FIG. 11 illustrates the representation of detected and classified irregular cardiac rhythms (i.e., episodes). Section 1101 displays all available classes. Section 1102 shows all detected irregular rhythms of a class that can be specified by clicking the class name in section 1101. The irregular rhythms include, but not limited to, Atrial Fibrillation, Atrial Flutter, Ventricular Fibrillation, Ventricular Flutter, Pause, Supraventricular Tachycardia, Ventricular Tachycardia, Supraventricular Bigeminy, Supraventricular Trigeminy, Ventricular Bigeminy, Ventricular Trigeminy, AV Block, and Patient-Triggered Event. 1106 is a floating window that shows detailed information for a specified irregular rhythm in section 1102. The information in 1106 includes, but not limited to, the class name, start time, duration and annotation text. 1103 shows a time series of ECG waveform for a channel that can specified in 1104. The selected irregular rhythm in 1102 is also shown in 1103. 1108 is a function bar to help a user to amplify the ECG waveform, adjust the baseline and zoom in x- or y-axis.

In certain embodiment, there are four interactive editing methods in FIG. 11 as follows:

(11.1) in section 1103, a user can adjust the start and end time points for any episode strip.

(11.2) in section 1102, user can delete a whole episode strip.

(11.3) in section 1103, user can add an ECG episode strip and name the added strip as one of known irregular rhythm classes or a customized class.

(11.4) in section 1106, user can modify the annotation text section for each ECG episode strip by rewriting the content.

All the editing operations (11.1) to (11.4) in FIG. 11 are through the cloud server 108 and updated the annotation records in 111 of FIG. 1. The information on the FIG. 11 is updated upon successful update on annotation records in 111.

For all of above editing operations, the actual data operations are all through the cloud server 108 to update the annotation records in 111, shown in FIG. 1. The client-side information and display are updated as soon as the server-side data operation is successfully completed. This client-server synchronization approach has the following benefits: reduce user's memory load on local computer since there is no need to transmit large amount of ECG data objects; minimize user's computing needs since these time-consuming actions are performed on the cloud server side; and prevent possible loss of data from a connection problem since user's most recent actions are simultaneously updated on ECG records.

### Computer & Hardware Implementation of Disclosure

It should initially be understood that the disclosure herein may be implemented with any type of hardware and/or software, and may be implemented using one or more servers. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present disclosure, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media.

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### Selected Embodiments

Although the above description and the attached claims disclose a number of embodiments of the present invention, other alternative aspects of the invention are disclosed in the following further embodiments.

Embodiment 1. A system for analyzing electrocardiogram (ECG) signals, the system comprising: at least one processor coupled with at least one memory that stores machine executable instructions, where execution of the instructions by the at least one processor causes the system to: receive the ECG signals acquired at an ECG device; process the ECG signals to output a set of classified ECG waveforms; process the set of classified ECG waveforms to output one or more ECG beat templates, each of the one or more ECG beat templates including a cluster of classified ECG waveforms; obtain an edited set of classified ECG waveforms and/or an edited set of ECG templates according to input from at least one user; and process the edited set of classified ECG waveforms and/or the edited set of ECG templates to output a cardiac rhythm classification; wherein the edited set of classified ECG waveforms and/or the edited set of ECG templates are viewed and/or modified by the at least one user.
Embodiment 2. The system of embodiment 1, wherein process the ECG signals to output a set of classified ECG waveforms includes identifying R-wave peaks in the ECG signals.
Embodiment 3. The system of embodiment 2, wherein process the ECG signals to output the set of classified ECG waveforms further includes inputting the ECG signals and the R-wave peaks into a first neural network, the first neural network trained according to a supervised learning algorithm.
Embodiment 4. The system of embodiment 2, wherein process the set of classified ECG waveforms to output one or more ECG beat templates includes inputting the set of classified ECG waveforms, and the R-wave peaks into a second neural network, the second neural network trained according to an unsupervised learning algorithm.
Embodiment 5. The system of embodiment 1, wherein process the ECG signals to output the set of classified ECG waveforms includes filtering the ECG signals.
Embodiment 6. The system of embodiment 1, wherein the at least one memory stores further instructions that when executed cause the at least one processor to: generate one or more first interactive graphical user interfaces based on the set of classified ECG waveforms and the one or more ECG beat templates; and display the one or more first interactive graphical user interfaces on a display, the one or more first interactive graphical user interfaces configured to provide a plurality of interactive editing operations on one or more of the classified ECG waveforms and/or the one or more ECG beat templates.
Embodiment 7. The system of embodiment 6, wherein the plurality of editing operations includes re-classification of one or more ECG waveforms in the set of classified ECG waveforms.
Embodiment 8. The system of embodiment 6, wherein the plurality of editing operations includes one or more template editing operations, the one or more template editing operation including splitting a given template and/or merging two selected templates.
Embodiment 9. The system of embodiment 6, wherein the plurality of editing operations includes one or more of template editing operations and ECG beat editing operations via one or more of an interactive histogram of R-R intervals, an interactive time series of R-R intervals, an interactive scatter plot of R-R intervals, and a graph of overlapping classified ECG waveforms.
Embodiment 10. The system of embodiment 1, wherein process the edited set of classified ECG waveforms and/or the edited set of ECG templates to output a cardiac rhythm classification includes inputting the edited set of classified ECG waveforms and the edited set of ECG templates into a third neural network, the third neural network trained to detect the irregular cardiac rhythms.
Embodiment 11. The system of embodiment 1, wherein the at least one memory stores further instructions that when executed cause the at least one processor to: generate a second interactive graphical user interface based on the edited set of classified ECG waveforms, the edited set of ECG templates, and the cardiac arrhythmias; and display the second interactive graphical user interface, the second interactive graphical user interface configured to provide a plurality of second interactive editing operations on a selected time series of ECG waveforms, the selected time series of ECG waveforms including an indication of the detected cardiac arrhythmia.
Embodiment 12. A system for analyzing electrocardiogram (ECG) signals, the system comprising: at least one processor coupled with at least one memory that stores machine executable instructions, where execution of the instructions by the at least one processor causes the system to: receive electrical signals from an ECG sensor; process the electrical signals to output one or more sets of ECG features; display the one or more sets of ECG features via a first interactive user interface; update one or more sets of ECG features according to a user input via the first interactive user interface; and process the updated one or more sets of ECG features to output a cardiac rhythm classification.
Embodiment 13. The system of embodiment 12, wherein the one or more sets of ECG features includes R-wave peaks.
Embodiment 14. The system of embodiment 12, wherein the one or more sets of ECG features includes classified ECG beat waveforms.
Embodiment 15. The system of embodiment 14, wherein the classified ECG beat waveforms are obtained according to a first neural network algorithm, the first neural network algorithm trained to classify ECG beats by a supervised learning process.
Embodiment 16. The system of embodiment 12, wherein the one or more sets of ECG features includes clustered ECG beat templates, each ECG beat templates including a cluster of classified ECG beat waveforms.
Embodiment 17. The system of embodiment 16, wherein the clustered ECG beat templates are obtained according to a second neural network algorithm, the second neural network algorithm trained to cluster ECG beat waveforms by an unsupervised learning process.
Embodiment 18. The system of embodiment 12, wherein process the updated one or more sets of ECG features to output the cardiac rhythm classification includes inputting the updated one or more sets of ECG features to a third neural network algorithm, the third neural network algorithm trained to classify cardiac rhythms.
Embodiment 19. The system of embodiment 12, wherein the first interactive user interface is configured to provide a plurality of interactive editing operations on one or more sets of ECG features.
Embodiment 20. The system of embodiment 12, wherein the at least one memory stores further instructions that when executed cause the at least one processor to: generate a second interactive user interface based on the updated one or more sets of ECG features; and display the second interactive user interface, the second interactive user interface configured to provide a plurality of second interactive editing operations on a selected time series of ECG waveforms, the selected time series of ECG waveforms including an indication of the cardiac rhythm classification.
Embodiment 21. A system for analyzing electrocardiogram (ECG) signals, the system comprising: at least one processor coupled with at least one memory that stores machine executable instructions, where execution of the instructions by the at least one processor causes the processor to: receive the ECG signals from an ECG device; process the ECG signals to generate one or more of R-wave peaks, classified ECG waveforms, and clustered ECG templates, each clustered ECG template including a cluster of the classified ECG waveforms; generate an annotation dataset including the one or more of the R-wave peaks, the classified ECG waveforms, and the clustered templates; generate an interactive user interface based on the annotated dataset; and responsive to receiving, via the interactive user interface, one or more modifications to the one or more of the R-wave peaks, the classified ECG waveforms, and the clustered templates, update the annotated dataset; and generate classification of cardiac rhythms based on the updated annotated dataset.
Embodiment 22. The system of embodiment 21, wherein the classified ECG waveforms is generated by inputting the ECG signals and the R-wave peaks into a first neural network, the first neural network trained according to a supervised learning algorithm.
Embodiment 23. The system of embodiment 21, wherein the one or more ECG beat templates is generated by inputting the classified ECG waveforms, and the R-wave peaks into a second neural network, the second neural network trained according to an unsupervised learning algorithm.
Embodiment 24. The system of embodiment 21, wherein process the ECG signals to output one or more of R-wave peaks, classified ECG waveforms, and clustered ECG templates, includes filtering the ECG signals.
Embodiment 25. The system of embodiment 21, wherein the first interactive graphical user interface is configured to provide a plurality of interactive editing operations on the one or more of the R-wave peaks, the classified ECG waveforms, and the one or more ECG beat templates.
Embodiment 26. The system of embodiment 25, wherein the wherein the plurality of editing operations includes adjusting R-wave peaks of one or more ECG waveforms of the classified waveforms.
Embodiment 27. The system of embodiment 25, wherein the plurality of editing operations includes beat re-classification of one or more ECG waveforms of the classified ECG waveforms.
Embodiment 28. The system of embodiment 25, wherein the plurality of editing operations includes one or more template editing operations, the one or more template editing operation including splitting a given template and/or merging two selected templates.
Embodiment 29. The system of embodiment 25, wherein the plurality of editing operations includes one or more of template editing operations and ECG beat editing operations via one or more of an interactive histogram of R-R intervals, an interactive time series of R-R intervals, an interactive scatter plot of R-R intervals, and a graph of overlapping classified ECG waveforms.
Embodiment 30. The system of embodiment 21, wherein generate classification of cardiac rhythms based on the updated annotated dataset includes inputting the updated annotated dataset into a third neural network, the third neural network trained to detect the cardiac arrhythmias.
Embodiment 31. The system of embodiment 21, wherein the at least one memory stores further instructions that when executed cause the at least one processor to: generate a second interactive graphical user interface based on the updated annotated dataset; and display the second interactive graphical user interface, the second interactive graphical user interface configured to provide a plurality of second interactive editing operations on a selected time series of ECG waveforms, the selected time series of ECG waveforms including an indication of the classification of cardiac rhythms.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A system for analyzing electrocardiogram (ECG) signals, the system comprising:
at least one processor coupled with at least one memory that stores machine executable instructions, where execution of the instructions by the at least one processor causes the system to:
receive the ECG signals acquired at an ECG device;
process the ECG signals to output a set of classified ECG waveforms;
process the set of classified ECG waveforms to output one or more ECG beat templates, each of the one or more ECG beat templates including a cluster of classified ECG waveforms;
obtain an edited set of classified ECG waveforms and/or an edited set of ECG templates according to input from at least one user; and
process the edited set of classified ECG waveforms and/or the edited set of ECG templates to output a cardiac rhythm classification;
wherein the edited set of classified ECG waveforms and/or the edited set of ECG templates are viewed and/or modified by the at least one user.

2. The system of claim 1, wherein process the ECG signals to output a set of classified ECG waveforms includes identifying R-wave peaks in the ECG signals.

3. The system of claim 2, wherein process the ECG signals to output the set of classified ECG waveforms further includes inputting the ECG signals and the R-wave peaks into a first neural network, the first neural network trained according to a supervised learning algorithm.

4. The system of claim 2, wherein process the set of classified ECG waveforms to output one or more ECG beat templates includes inputting the set of classified ECG waveforms, and the R-wave peaks into a second neural network, the second neural network trained according to an unsupervised learning algorithm.

5. The system of claim 1, wherein process the ECG signals to output the set of classified ECG waveforms includes filtering the ECG signals.

6. The system of claim 1, wherein the at least one memory stores further instructions that when executed cause the at least one processor to:
generate one or more first interactive graphical user interfaces based on the set of classified ECG waveforms and the one or more ECG beat templates; and
display the one or more first interactive graphical user interfaces on a display, the one or more first interactive graphical user interfaces configured to provide a plurality of interactive editing operations on one or more of the classified ECG waveforms and/or the one or more ECG beat templates.

7. The system of claim 6, wherein the plurality of editing operations includes re-classification of one or more ECG waveforms in the set of classified ECG waveforms.

8. The system of claim 6, wherein the plurality of editing operations includes one or more template editing operations, the one or more template editing operation including splitting a given template and/or merging two selected templates.

9. The system of claim 6, wherein the plurality of editing operations includes one or more of template editing operations and ECG beat editing operations via one or more of an interactive histogram of R-R intervals, an interactive time series of R-R intervals, an interactive scatter plot of R-R intervals, and a graph of overlapping classified ECG waveforms.

10. The system of claim 1, wherein process the edited set of classified ECG waveforms and/or the edited set of ECG templates to output a cardiac rhythm classification includes inputting the edited set of classified ECG waveforms and the edited set of ECG templates into a third neural network, the third neural network trained to detect the irregular cardiac rhythms.

11. The system of claim 1, wherein the at least one memory stores further instructions that when executed cause the at least one processor to:
generate a second interactive graphical user interface based on the edited set of classified ECG waveforms, the edited set of ECG templates, and the cardiac arrhythmias; and
display the second interactive graphical user interface, the second interactive graphical user interface configured to provide a plurality of second interactive editing operations on a selected time series of ECG waveforms, the selected time series of ECG waveforms including an indication of the detected cardiac arrhythmia.

12. A system for analyzing electrocardiogram (ECG) signals, the system comprising:
at least one processor coupled with at least one memory that stores machine executable instructions, where execution of the instructions by the at least one processor causes the system to:
receive electrical signals from an ECG sensor;
process the electrical signals to output one or more sets of ECG features;
display the one or more sets of ECG features via a first interactive user interface;
update one or more sets of ECG features according to a user input via the first interactive user interface; and
process the updated one or more sets of ECG features to output a cardiac rhythm classification.

13. The system of claim 12, wherein the one or more sets of ECG features includes R-wave peaks.

14. The system of claim 12, wherein the one or more sets of ECG features includes classified ECG beat waveforms.

15. The system of claim 14, wherein the classified ECG beat waveforms are obtained according to a first neural network algorithm, the first neural network algorithm trained to classify ECG beats by a supervised learning process.

16. The system of claim 12, wherein the one or more sets of ECG features includes clustered ECG beat templates, each ECG beat templates including a cluster of classified ECG beat waveforms.

17. The system of claim 16, wherein the clustered ECG beat templates are obtained according to a second neural network algorithm, the second neural network algorithm trained to cluster ECG beat waveforms by an unsupervised learning process.

18. The system of claim 12, wherein process the updated one or more sets of ECG features to output the cardiac rhythm classification includes inputting the updated one or more sets of ECG features to a third neural network algorithm, the third neural network algorithm trained to classify cardiac rhythms.

19. The system of claim 12, wherein the first interactive user interface is configured to provide a plurality of interactive editing operations on one or more sets of ECG features.

20. The system of claim 12, wherein the at least one memory stores further instructions that when executed cause the at least one processor to:
generate a second interactive user interface based on the updated one or more sets of ECG features; and
display the second interactive user interface, the second interactive user interface configured to provide a plurality of second interactive editing operations on a selected time series of ECG waveforms, the selected time series of ECG waveforms including an indication of the cardiac rhythm classification.

21. A system for analyzing electrocardiogram (ECG) signals, the system comprising:
at least one processor coupled with at least one memory that stores machine executable instructions, where execution of the instructions by the at least one processor causes the processor to:
receive the ECG signals from an ECG device;
process the ECG signals to generate one or more of R-wave peaks, classified ECG waveforms, and clustered ECG templates, each clustered ECG template including a cluster of the classified ECG waveforms;
generate an annotation dataset including the one or more of the R-wave peaks, the classified ECG waveforms, and the clustered templates;
generate an interactive user interface based on the annotated dataset; and
responsive to receiving, via the interactive user interface, one or more modifications to the one or more of the R-wave peaks, the classified ECG waveforms, and the clustered templates,
update the annotated dataset; and
generate classification of cardiac rhythms based on the updated annotated dataset.

22. The system of claim 21, wherein the classified ECG waveforms is generated by inputting the ECG signals and the R-wave peaks into a first neural network, the first neural network trained according to a supervised learning algorithm.

23. The system of claim 21, wherein the one or more ECG beat templates is generated by inputting the classified ECG waveforms, and the R-wave peaks into a second neural network, the second neural network trained according to an unsupervised learning algorithm.

24. The system of claim 21, wherein process the ECG signals to output one or more of R-wave peaks, classified ECG waveforms, and clustered ECG templates, includes filtering the ECG signals.

25. The system of claim 21, wherein the first interactive graphical user interface is configured to provide a plurality of interactive editing operations on the one or more of the R-wave peaks, the classified ECG waveforms, and the one or more ECG beat templates.

26. The system of claim 25, wherein the wherein the plurality of editing operations includes adjusting R-wave peaks of one or more ECG waveforms of the classified waveforms.

27. The system of claim 25, wherein the plurality of editing operations includes beat re-classification of one or more ECG waveforms of the classified ECG waveforms.

28. The system of claim 25, wherein the plurality of editing operations includes one or more of template editing operations and ECG beat editing operations via one or more of an interactive histogram of R-R intervals, an interactive time series of R-R intervals, an interactive scatter plot of R-R intervals, and a graph of overlapping classified ECG waveforms.

29. The system of claim 21, wherein generate classification of cardiac rhythms based on the updated annotated dataset includes inputting the updated annotated dataset into a third neural network, the third neural network trained to detect the cardiac arrhythmias.

30. The system of claim 21, wherein the at least one memory stores further instructions that when executed cause the at least one processor to:
generate a second interactive graphical user interface based on the updated annotated dataset; and
display the second interactive graphical user interface, the second interactive graphical user interface configured to provide a plurality of second interactive editing operations on a selected time series of ECG waveforms, the selected time series of ECG waveforms including an indication of the classification of cardiac rhythms.
